(19) United States
(12) Patent Application Publication
(10) Pub. No.: US 2016/0282698 A1
(43) Pub. Date: Sep. 29, 2016

(54) HIGH INDEX CONTRAST WAVEGUIDE DEVICES AND SYSTEMS

(71) Applicant: Intel Corporation, Santa Clara, CA (US)

(72) Inventor: Bruce A. Block, Portland, OR (US)

(21) Appl. No.: 14/670,217

(22) Filed: Mar. 26, 2015

(51) Int. Cl. G02F 1/225; A61B 5/0492; A61B 5/0478; A61B 5/0496; G01R 29/08; A61B 5/0408

(52) U.S. Cl. CPC G02F 1/225; G01R 29/0814; A61B 5/0408; A61B 5/0478; A61B 5/0496; A61B 5/0492; G02F 2001/212

(57) ABSTRACT

A high index contrast waveguide device is disclosed and described. In one embodiment the waveguide may include a lithium niobate substrate, a waveguide with a higher refractive index than that of the lithium niobate substrate patterned on a surface thereof, and an electrode electrically coupled to the waveguide.

### BACKGROUND

Lithium niobate (LiNbO3) is an optically active material having an index of refraction that varies as a function of applied electrical energy. Lithium niobate has been previously used in waveguide devices, which generally include a lithium niobate substrate with a waveguide material disposed within the substrate. Common techniques for making such waveguides include titanium diffusion or annealed proton exchange processes to create a graded index. The index of refraction of such graded indexes vary as a function of the distance from the waveguide center axis. In such cases, the index contrast between the waveguide and any overlying cladding is very small, and thus the optical mode size is relatively large (e.g. about 8-10 microns).

### BRIEF DESCRIPTION OF THE DRAWINGS

FIG. 1 is a schematic view of a waveguide device in accordance with an invention embodiment;

FIG. 2 is a schematic view of a waveguide device in accordance with an invention embodiment;

FIG. 3 is a schematic view of a waveguide device in accordance with an invention embodiment;

FIG. 4 is a schematic view of a waveguide device in accordance with an invention embodiment;

FIG. 5 is a schematic view of a sensor system in accordance with an invention embodiment;

FIG. 6A is a graphical depiction of simulation data in accordance with an invention embodiment; and

FIG. 6B is a graphical depiction of simulation data in accordance with an invention embodiment.

### DESCRIPTION OF EMBODIMENTS

Although the following detailed description contains many specifics for the purpose of illustration, a person of ordinary skill in the art will appreciate that many variations and alterations to the following details can be made and are considered to be included herein.

Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this specification and the appended claims, the singular forms “a,” “an” and “the” include plural referents unless the context clearly dictates otherwise. Thus, for example, reference to “a sensor” includes a plurality of such sensors.

In this specification, “comprises,” “comprising,” “containing” and “having” and the like can have the meaning ascribed to them in U.S. patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. patent law. “Consisting essentially of” or “consists essentially of” have the meaning generally ascribed to them by U.S. patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly and vice versa.

“The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein. The term “coupled,” as used herein, is defined as directly or indirectly connected in an electrical or nonelectrical manner. Objects or structures described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, the term “high index contrast” in the context of lithium niobate waveguides refers to index contrasts (Δn) greater than 0.1.

As used herein, “enhanced,” “improved,” “performance-enhanced,” “upgraded,” and the like, when used in connection with the description of a device or process, refers to a characteristic of the device or process that provides measurably better form or function as compared to previously known devices or processes. This applies both to the form and function of individual components in a device or process, as well as to such devices or processes as a whole.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. However, it is to be understood that even when the term “about” is used in the present specification in connection with a specific numerical value, that support for the exact numerical value recited apart from the “about” terminology is also provided.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### Example Embodiments

An initial overview of technology embodiments is provided below and specific technology embodiments are then described in further detail. This initial summary is intended to aid readers in understanding the technology more quickly but is not intended to identify key or essential technological features, nor is it intended to limit the scope of the claimed subject matter.

The present disclosure provides high index contrast waveguide devices and systems. Such waveguide devices can be designed to have significantly lower power consumptions and significantly smaller form factors compared to traditional waveguide devices, as well as having markedly increased performance characteristics. Traditional lithium niobate (LiNbO₃) waveguide devices, for example, generally include a lithium niobate substrate with a waveguide material disposed within the substrate. In some cases, such a waveguide can be made by titanium diffusion or annealed proton exchange processes to create a graded index. Thus, the index of refraction of the waveguide varies as a function of the distance from the waveguide center axis. In such cases, the index contrast between the waveguide and any overlying cladding is very small, and the optical mode size is relatively large (e.g. about 8-10 microns). In order to turn light, such low index contrast waveguides require large bend radii to reduce scatter loss, and gradual splitters are often used to reduce optical loss for devices such as Mach Zehnder interferometers (MZI). Additionally, as the mode size of these devices is large, wide gaps must be used between electrodes in order to minimize optical loss. 10-15 micron gaps are typically used in the art. As a result, traditional high-speed optical modulators based on lithium niobate MZI interferometers are typically several centimeters long and require travelling wave electrodes to achieve high data rates with moderate voltage (e.g. 3-7 Vpp). Since the optical and electrical fields travel at different rates, such traditional lithium niobate modulators are limited to speeds of less than about 40 Gb/s. The long lengths of such devices also negatively affect performance as the optical insertion loss is relatively high, in some cases from about 5 to 8 dB.

By comparison, high index contrast waveguide devices and systems as disclosed herein include a waveguide material formed or otherwise disposed on a surface of a lithium niobate substrate. This waveguide material has a high refractive index, which is higher than the refractive index of lithium niobate. Such a design can significantly reduce the optical mode size of the waveguide on the lithium niobate substrate as compared to the optical mode size of a traditional waveguide device. As a result of the reduced optical mode size, electromagnetic radiation can be more effectively confined in the waveguide, thus contributing to an increase in the optical efficiency of the device. The dimensions of the waveguide can also be designed such that there is a large portion of the evanescent tail of the optical waveguide mode that resides in the lithium niobate substrate.

The optical mode size represents the physical dimensions of the optical mode, or in other words, the cross-sectional average diameter of the optical mode. This can be determined from the dimensions of the waveguide and the index contrast, as a larger index contrast results in a smaller optical mode size. Reducing the optical mode size allows electrodes to be positioned closer to the waveguide without incurring optical loss from light contacting the electrodes, because the evanescent tail of the optical mode typically limits electrode positioning. As an example, a particular waveguide having an optical mode size of about 1 micron may require electrodes to be positioned further apart than one micron if the evanescent tail of the optical mode extends two microns into the lithium niobate substrate. It is noted that, for values presented herein, optical mode size is for the transverse electric field (TE); however, those skilled in the art can readily convert or otherwise determine optical mode size for other modes such as, for example, transverse magnetic (TM) mode.

Optical mode size of high index contrast devices according to the presently disclosed technology can generally be less than 8 microns. In one example, the optical mode can be less than 3 microns. In another example, the optical mode can be from 1 micron to 3 microns.

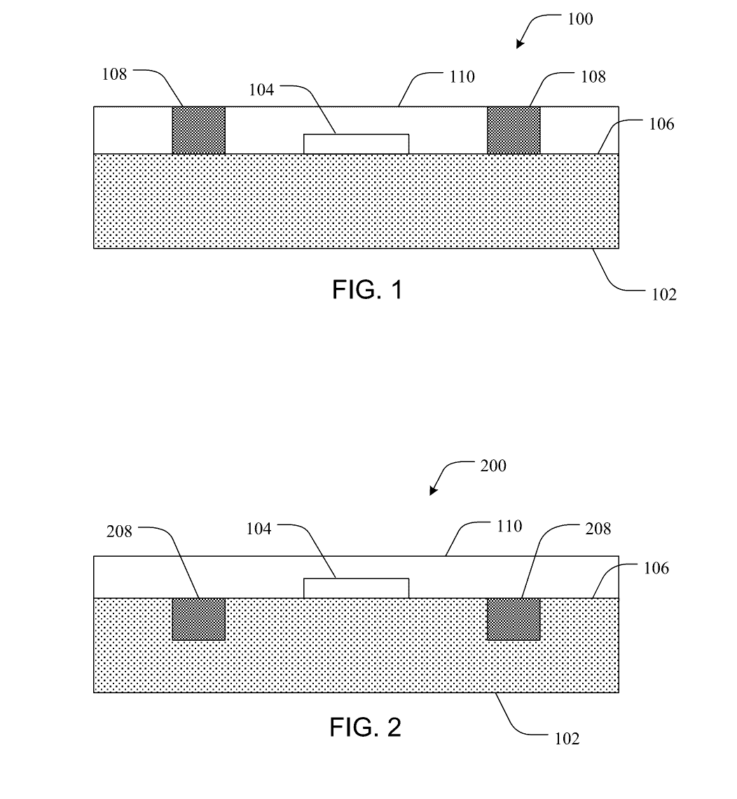

One exemplary high index contrast waveguide device is shown in FIG. 1. Device 100 includes a lithium niobate substrate 102 and a waveguide 104 patterned or otherwise disposed on a surface 106 of the lithium niobate substrate 102. The waveguide further has a refractive index that is higher than the refractive index of the lithium niobate substrate 102. Device 100 also includes an electrode 108 electrically coupled to the waveguide 104, and a cladding layer 110 disposed on the surface 106. In some examples a device can include a plurality of electrodes, as is shown in FIG. 1. The electrodes 108 shown in FIG. 1 are disposed on the surface 106 of the lithium niobate substrate 102. FIG. 2 shows another example of a high index contrast waveguide device 200 similar to FIG. 1, but where the electrodes 208 are disposed or otherwise recessed within the lithium niobate substrate 108. Further examples are contemplated in which an electrode may be only partially recessed into the lithium niobate substrate, and thus would have an intermediate protrusion from the substrate between what is shown in FIG. 1 and FIG. 2. Also, in devices having multiple electrodes, all electrodes can be positioned at the same protrusion, or electrodes can be positioned at different protrusions depending on the design of the device. Regarding spacing, in one example electrodes can be spaced from at least 1 micron apart up to a functional limit imposed by the materials and design of the device. In another example, electrodes can be spaced from 1 micron up to 10 microns apart. In yet another example, electrodes can be spaced from 1.5 microns up to 5 microns apart.

Additionally, while FIGS. 1 and 2 show a single waveguide 104, in some embodiments multiple waveguides can be utilized. Multiple waveguides can include multiple discrete waveguides, as well as multiple waveguide segments joined together by a waveguide bend, splitter, or other waveguide structure to form a continuous optical pathway. Thus, a given device design can vary, and the present scope is intended to cover any design that incorporates a waveguide disposed on a surface of a lithium niobate substrate.

Numerous waveguide materials are contemplated, and any material capable of being used to create a waveguide device according to the present disclosure is considered to be within the present scope. For example, the waveguide material can be any material capable of being used as a waveguide that has an index of refraction greater than the index of refraction of lithium niobate. As another example, the waveguide material can be any material capable of being used as a waveguide that has an index of refraction of greater than 2.4. Non-limiting examples can include silicon materials, such as amorphous silicon, polycrystalline silicon, a-SiC and the like, various oxides such as titanium dioxide, chalcogenides, and the like. Depending on light wavelength, good quality semiconductor materials having wavelength energy below the bandgap can also be used. As one example, germanium could be used for light wavelengths longer than about 1.6 microns.

Additionally the substrate can include or be made from a number of electro-optic materials aside from the lithium niobate, such as lithium tantalate, β-BaB2O4 (BBO), and other known materials which function, or can be made to function as the substrate. Further, the cladding material used for the cladding can be any suitable low refractive index material know for use in waveguides.

The physical dimensions of the waveguide can vary depending on the device design, the nature of the selected materials, intended use of the resulting device, and the like. The thickness of the waveguide, for example, is at least partially dependent on the index of refraction of the waveguide material. It may be beneficial, in some cases, for the thickness and width of the waveguide to be chosen to provide a single mode waveguide. Larger waveguides having more than one mode can lower the efficiency of the waveguide device. Generally, the efficiency of a given device will be higher for a smaller cross-sectional area waveguide. If the cross-sectional area is too small, however, either the optical mode will not be supported, or the optical mode will expand such that it will be weakly confined, and thus will behave as a low index contrast waveguide. One of ordinary skill in the art, once in possession of the present disclosure, can readily calculate appropriate physical waveguide dimensions. As one example using 1310 nm light, a waveguide of amorphous silicon can be from 80 nm to 220 nm thick, and can have a width of from 400 nm to 1000 nm. In another example using 1310 nm light, a waveguide of titanium dioxide can be from 400 nm to 1000 nm thick and have a width of from 400 nm to 1000 nm. A material having a refractive index between that of silicon and titanium dioxide may have intermediate waveguide dimensions. As further 1310 nm light examples, a waveguide of amorphous silicon can be from 80 nm to 120 nm thick and 400 nm to 1000 nm wide, and a waveguide of titanium dioxide can be from 400 nm to 500 nm thick and from 400 nm to 600 nm wide.

It is additionally noted that the dimensions of a given waveguide can be adjusted for various reasons to suit a desired outcome. For example, narrowing the width along a straight portion of a waveguide can increase efficiency. As another example, a waveguide can be widened at a bend in the waveguide to lower optical loss at the bend.

As has been described above, the presently described waveguide technology allows a waveguide to be formed having a bend or turn radius that is greatly reduced as compared to traditional waveguide devices, which can often be several millimeters. For example, a bend radius of a waveguide according to the present disclosure can be as small as 5 microns or less, depending on the physical dimensions of the waveguide. In one example, a bend radius measured from the inside radius of a waveguide can be from 5 microns up to a radius that is equivalent to a straight waveguide section. In another example, a bend radius measured from the inside radius of a waveguide can be from 5 microns up to 50 microns. In yet another example, a bend radius measured from the inside radius of a waveguide can be from 5 microns up to 30 microns, or 5 microns up to 10 microns. In another example, a bend radius measured from the inside radius of a waveguide can be from 25 microns up to 50 microns, or from 20 microns up to 30 microns. It is noted that for tighter turn radii, such as below 20 microns for example, it may be necessary to thicken the waveguide, which can result in decreased efficiency. This effect can be mitigated by adiabatically increasing the waveguide width prior to the bend to improve bend loss then adiabatically decreasing the width after the bend.

The small bend radii achieved by the presently disclosed technology can greatly enhance the performance of a waveguide device, as well as allowing for a greatly decreased device footprint. Such small bend radii enable compacting of the device via folds to reduce the footprint of the device. A small footprint can eliminate the need for travelling wave electrodes that can limit the operating frequency in a lithium niobate modulator and thus significantly lower the device power. Thus, in addition to the benefits of smaller-sized devices, power consumption can be decreased through such compacting. The footprint of a given device can be made even smaller through the use of a ring resonator, which would not be practical in a traditional lithium niobate waveguide device. Furthermore, as the length of a waveguide device is decreased, the optical loss is decreased as well, which results in a beneficial signal to noise increase.

While there effectively no limits on how large a footprint of a presently disclosed waveguide device can be, in one example a footprint can be as small as 1 mm×1 mm, or where at least one width of the footprint is as small as 1 mm. In other examples, a footprint can be as small as 2 mm×2 mm, or 5 mm×5 mm, or where a minimum width can be 2 mm, or 5 mm.

As has also been described above, the efficiency of the presently disclosed waveguide devices is greatly increased compared to traditional devices. Generally, the efficiency can be defined as the ratio of the electrically controlled index of refraction change in the lithium niobate to the effective index of refraction change of the waveguide. This efficiency can be measured by applying a known voltage to the device and measuring the resulting intensity change. Using the electro-optic coefficients of the waveguide material, the efficiency can be calculated. In one example, a waveguide device can have an efficiency of greater than 40%, greater than 70%, or greater than 75%. In another aspect, a waveguide device can have an efficiency of from 40% to 75%. An additional way of considering efficiency may be in terms of the change in Δn_{eff} with applied voltage. In a waveguide the effective mode index change Δn_{eff} is the key value given by: Δn_{eff}=½Γ n³rE. The Γ for a traditional lithium niobate device can range from about 50% to about 75%. Because the electrode gap for high index waveguide devices is small, the voltage is also small while the electric field is higher. Thus, a higher Δn_{eff} is achieved.

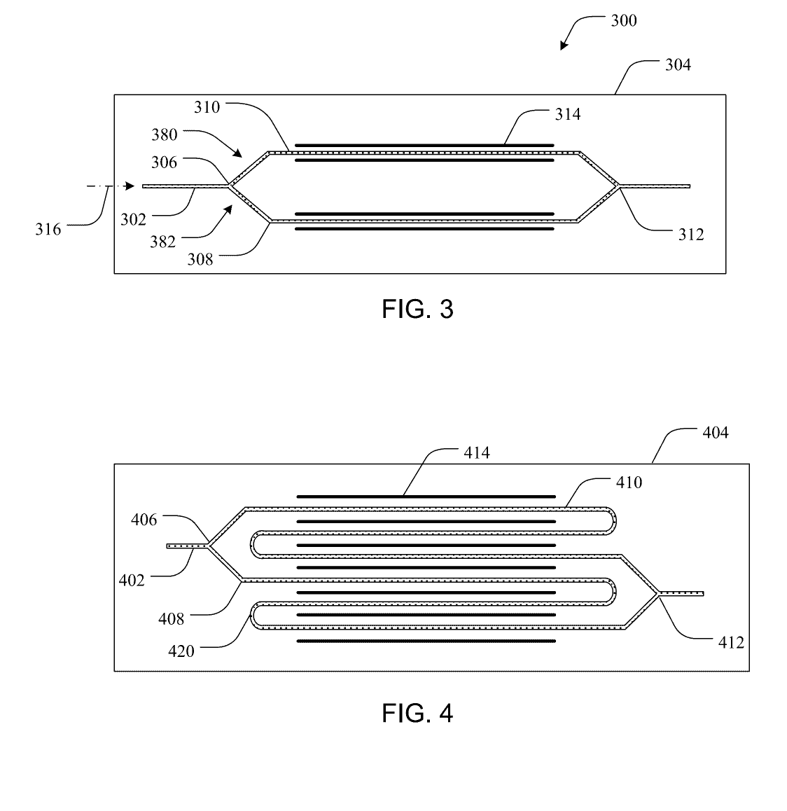

Waveguide devices can be used in a wide variety of applications that can benefit from reduced size and enhanced performance characteristics. Many applications utilize some form of interferometry, such as the commonly-used Mach Zehnder Interferometer (MZI). FIG. 3 shows a non-limiting example of a MZI configuration for a waveguide device 300 utilizing the presently disclosed high index contrast waveguide technology. A waveguide 302 is shown disposed on a surface of a lithium niobate substrate 304. The waveguide 302 is split into two optical pathways 380, 382, by a waveguide splitter 306. Device 300 can include any number of waveguide bends 308 and waveguide straights 310 depending on the waveguide device design. A waveguide re-combiner 312 can be positioned to receive light from multiple optical pathways, and is operable to combine received light into a single optical output. Electrodes can be functionally coupled to the waveguide, and can be positioned in a variety of configurations. In the case of FIG. 3, electrodes 314 are functionally coupled to each optical pathway, and for each pathway are positioned on either side of the waveguide straights 310. The waveguide device receives light 316 from a light source (not shown), which travels along the waveguide 302, through the waveguide splitter 306, along the two optical pathways 380, 382, and through the waveguide re-combiner 312 to be recombined into a common resultant beam. A phase difference between the two pathways results in a phase modulation in the resultant beam. This effect can be utilized for various types of applications, including both modulation and detection. Furthermore, the presently disclosed technology can be utilized in various devices, including, without limitations, ring and microring resonators, photonic band structure devices, thin film lithium niobate substrate devices, and the like.

For modulation of an optical signal, an electrical voltage applied across an electrode pair associated with an optical pathway can alter the index of refraction of the lithium niobate material and thus the index contrast of that pathway, thus generating a phase change in the light passing through that optical pathway. By driving the electrodes associated with one or both of the optical pathways, a modulated signal can be generated from an optical waveform. Such a modulated signal can be used for communication at very high bit rates, and can be used in a wide variety of applications in, for example, the telecom and datacom fields.

Additionally, such devices can also be used as optical electric field sensors. By electrically coupling electrodes across an electric potential to be measured, optical intensity is varied in the resultant beam due to the changes in the index of refraction, as was described for modulator configurations. Such variations can be utilized to detect, monitor, and/or quantify aspects of an electric field.

FIG. 4 shows a folded MZI design that can further decrease the footprint of a high index contrast device 400. Similar to the design shown in FIG. 3, the waveguide 402 of device 400 can include various waveguide splitters 406, waveguide bends 408, waveguide straights 410, and waveguide re-combiners 412 disposed on a lithium niobate substrate 404. The optical pathways shown are thus folded back on one another using waveguide bends (or turns) 420. Such a folded configuration allows electrodes 414 to be shared between folded portions, thus reducing the total electrode number. This, combined with the smaller gap spacing between electrodes afforded by the disclosed high index contrast waveguide technology, allows the overall device footprint to be greatly reduced.

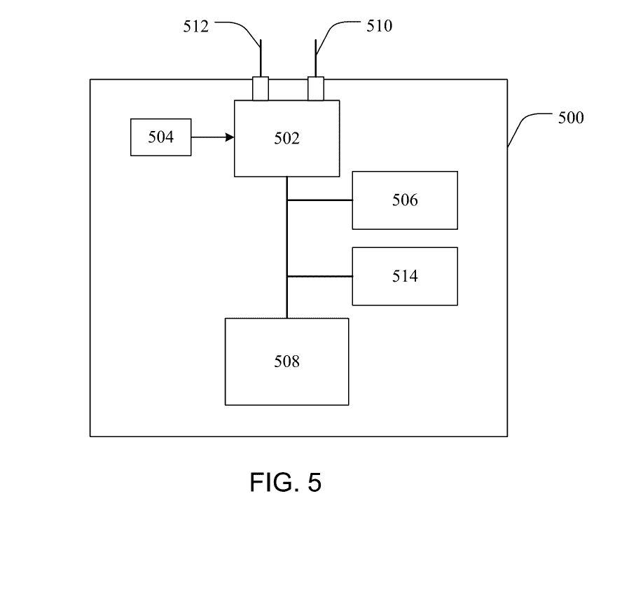

One example of a system or sensor device, such as an integrated device for sensing an electric field is shown in FIG. 5. Such a system or device 500 can include a sensing device or module shown generally as box 502. The sensing device or module can include a lithium niobate substrate, a waveguide patterned on a surface of the lithium niobate substrate, a first electrode positioned adjacent to the waveguide, and a second electrode positioned adjacent to the waveguide on an opposite side from the first electrode. The waveguide has a refractive index that is higher than a refractive index of the lithium niobate substrate, as has been described herein. System 500 can include a light source 504 optically coupled to the sensing device or module 502 such that light from the light source 504 is delivered into the waveguide. The light source can include any light source capable of generating coherent light usable in a waveguide device according to the present disclosure. Non-limiting examples can include laser sources, laser diodes, and the like.

System or integrated device 500 can further include an analytic module 506 functionally coupled to the first and second electrode, which can operate to determine an electrical potential across the first and second electrodes. Determination can include the detection, monitoring, and/or quantification of the electric field. A power source 508 is electrically coupled to the analytic module 506 and to the first and second electrodes of the sensing device 502. A first measurement electrode 510 and a second measurement electrode 512 are electrically coupled to the first and second electrodes respectively, and operate to relay electric field measurements from an environment external to the system to the first and second electrodes. It is noted that the term “measurement electrode” can include an electrode for making electric field measurements, as well as an electrical connector for coupling such an electrode thereto. Measurement electrodes can include a wide variety of electrode designs depending on the electric field, the nature of the testing, and environment in which testing is to occur. Measurement electrodes can be wet contact, dry contact, or contactless electrodes.

Returning to FIG. 5, an indicator 514 can be functionally coupled to the analytic module 506 and operate to indicate electric field measurements to a user. The indicator 514 can be any type of indicator, including without limitation audible indicators, illumination indicators, visual display indicators, and the like. The indicator 514 can also be a signal output for communication with a separate electronic device. Thus an indication signal can include audible signals, visual signals, electronic signals, and the like.

In some examples, the measurement electrodes can be designed to functionally couple to a biological entity, including humans, non-human mammals, reptiles, birds, or any other biological entity capable of generating an electric field, including plant matter, cellular cultures, tissues, and the like. Measurement electrodes thus can operate to relay electric field measurements from an electric field generated by a biological entity to the sensing device 502 of the system 500. Non-limiting examples of such electric fields can include electrocardiogram signals, electroencephalogram signals, electromyocardium signals, electrooculogram signals, including combinations thereof.

Additionally, the increased sensitivity and efficiency of the presently disclosed devices and systems allow the detection of electric fields at much higher frequencies compared to traditional sensor devices. For example, such devices can be utilized to detect RF signals or fields.

In addition to devices and systems there is further provided methods of detecting or measuring an electric field. At a basic level, such a method can include providing a waveguide device or system as recited herein, placing measurement electrodes of the device or system in a position operable to receive input from an electric field, and operating the device, which can include inputting a light source into the waveguide and detecting variations in the light at a detector after passing between electrodes that are electrically coupled to the measuring electrodes. Gathering and quantification of the measured information can then be done using any suitably known devices, components, or modules in order to provide a user with information concerning the detection or measurement of the electric field.

### Examples

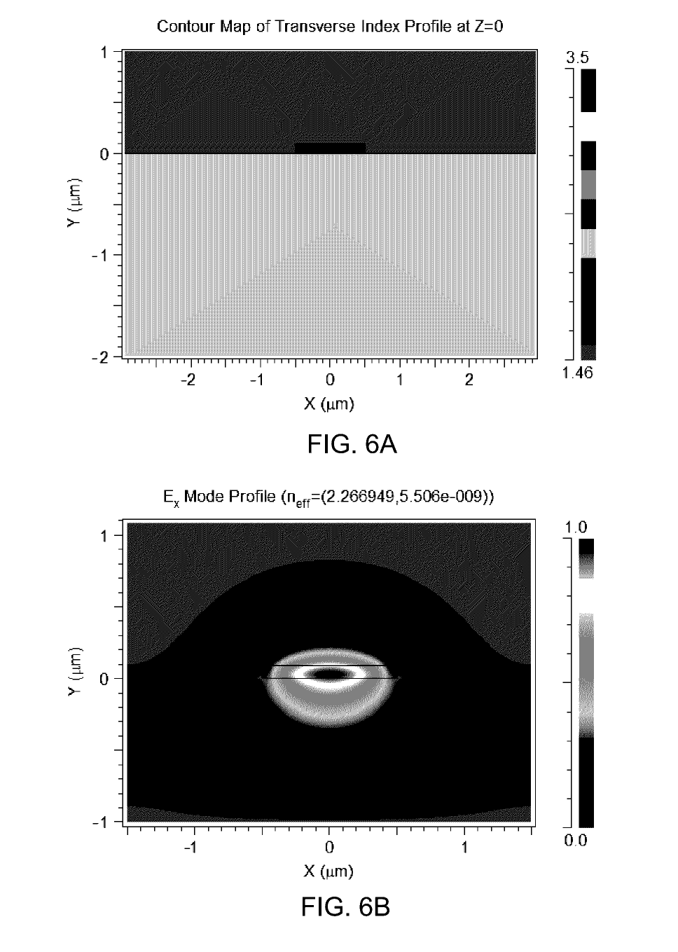

FIGS. 6A and 6B show simulation data generated for 1.55 micron light for a 90 nm×1000 nm amorphous silicon waveguide on a lithium niobate substrate. FIG. 6A shows a contour map of the transverse index profile at Z=0, and FIG. 6B shows the simulated optical mode in relation to the amorphous silicon waveguide.

In one exemplary embodiment there is provided a high index contrast waveguide device, comprising:

a lithium niobate substrate;

a waveguide patterned on a surface of the lithium niobate substrate, the waveguide having a refractive index that is higher than a refractive index of the lithium niobate substrate; and

an electrode electrically coupled to the waveguide.

In one example, the waveguide includes amorphous silicon, polysilicon, titanium dioxide, or a combination thereof.

In one example, the waveguide includes a material having an index of refraction of greater than 2.4.

In one example, the waveguide includes at least one turn radius of from 5 microns to 50 microns measured at an inside edge of the waveguide.

In one example, the waveguide includes at least one turn radius of from 20 microns to 30 microns measured at an inside edge of the waveguide.

In one example, the waveguide includes at least one turn radius of from 5 microns to 100 microns measured at an inside edge of the waveguide.

In one example, the device has an efficiency of greater than 70%.

In one example, the device has an efficiency of from about 40% to about 75%.

In one example, the device has an optical mode size of less than 3 microns.

In one example, the device has an optical mode size of from about 1 micron to about 3 microns.

In one example, the electrode includes a pair of electrodes.

In one example, the electrodes in an electrode pair have an electrode-to-electrode spacing of from about 1 micron to about 10 microns.

In one example, the electrodes in an electrode pair have an electrode-to-electrode spacing of from about 1.5 micron to about 5 microns.

In one example, the electrodes of an electrode pair are positioned on opposite sides of the at least one waveguide.

In one example, the electrode is disposed on a surface of the lithium niobate substrate.

In one example, the electrode is recessed into a surface of the lithium niobate substrate.

In one example, the electrode is completely recessed into the surface of the lithium niobate substrate.

In one example, the waveguide is a pair of waveguides and the electrode is a pair of electrodes, and wherein each waveguide in a pair of waveguides is disposed between electrodes of an electrode pair.

In one example, the pair of waveguides and the pair of electrodes are configured as a Mach Zehnder interferometer.

In one example, the pair of waveguides and the pair of electrodes are configured as an optical modulator.

In one example, the pair of waveguides and the pair of electrodes are configured as an electric field sensor.

In one example, the device is configured as a ring resonator device.

In one example, the device is configured as a photonic band structure device.

In one example, the device further comprises a cladding layer disposed on and covering the lithium niobate substrate and the waveguide.

In an exemplary embodiment there is provided a system for sensing an electric field, comprising:

a sensing device comprising;

a lithium niobate substrate;

a waveguide disposed on a surface of the lithium niobate substrate, wherein the waveguide has a refractive index that is higher than a refractive index of the lithium niobate substrate;

a first electrode positioned adjacent to the waveguide; and

a second electrode positioned adjacent to the waveguide on an opposite side from the first electrode;

a light source optically coupled to the sensing device and positioned to deliver light into the waveguide;

an analytic module functionally coupled to the first and second electrode and operable to determine an electrical potential across the first and second electrodes;

a power source electrically coupled to the analytic module and to the first and second electrodes;

a first measurement electrode and a second measurement electrode electrically coupled respectively to the first and second electrodes, and operable to relay electric field measurements from an environment external to the system to the first and second electrodes; and

an indicator functionally coupled to the analytic module and operable to indicate the electric field measurements to a user.

In one example, the analytic module is operable to quantify the electric field measurements.

In one example, the indicator generates an audible signal.

In one example, the indicator generates a visual signal.

In one example, the indicator generates an electronic signal.

In one example, the indicator is a display screen.

In one example, the first and second measurement electrodes are dry contact electrodes.

In one example, the first and second measurement electrodes are contactless electrodes.

In one example, the first and second measurement electrodes are operable to functionally couple to a biological entity.

In one example, the first and second measurement electrodes are operable to relay electric field measurements from an electric field generated by a biological system of the biological entity.

In one example, the electric field is an electrocardiogram signal, an electroencephalogram signal, an electromyocardium signal, an electrooculogram signal, or a combination thereof.

In one example, the first and second measurement electrodes are operable to relay RF electric field measurements.

While the forgoing examples are illustrative of the specific embodiments in one or more particular applications, it will be apparent to those of ordinary skill in the art that numerous modifications in form, usage and details of implementation can be made without departing from the principles and concepts articulated herein. Accordingly, no limitation is intended except as by the claims set forth below.

What is claimed is:

1. A high index contrast waveguide device, comprising:
a lithium niobate substrate;
a waveguide patterned on a surface of the lithium niobate substrate, the waveguide having a refractive index that is higher than a refractive index of the lithium niobate substrate; and
an electrode electrically coupled to the waveguide.

2. The device of claim 1, wherein the waveguide includes amorphous silicon, polysilicon, titanium dioxide, or a combination thereof.

3. The device of claim 1, wherein the waveguide includes a material having an index of refraction of greater than 2.4.

4. The device of claim 1, wherein the waveguide includes at least one turn radius of from 5 microns to 50 microns measured at an inside edge of the waveguide.

5. The device of claim 1, wherein the device has an efficiency of from about 40% to about 75%.

6. The device of claim 1, wherein the device has an optical mode size of from about 1 micron to about 3 microns.

7. The device of claim 1, wherein the electrode includes a pair of electrodes.

8. The device of claim 7, wherein electrodes in the electrode pair have an electrode-to-electrode spacing of from about 1 micron to about 10 microns.

9. The device of claim 7, wherein electrodes of the electrode pair are positioned on opposite sides of the at least one waveguide.

10. The device of claim 1, wherein the electrode is disposed on a surface of the lithium niobate substrate.

11. The device of claim 1, wherein the electrode is recessed into a surface of the lithium niobate substrate.

12. The device of claim 11, wherein the electrode is completely recessed into the surface of the lithium niobate substrate.

13. The device of claim 1, wherein the waveguide is a pair of waveguides and the electrode is a pair of electrodes, and wherein each waveguide in a pair of waveguides is disposed between electrodes of an electrode pair.

14. The device of claim 13, wherein the pair of waveguides and the pair of electrodes are configured as an electric field sensor.

15. The device of claim 1, further comprising a cladding layer disposed on and covering the lithium niobate substrate and the waveguide.

16. A system for sensing an electric field, comprising:
a sensing device comprising;
a lithium niobate substrate;
a waveguide disposed on a surface of the lithium niobate substrate, wherein the waveguide has a refractive index that is higher than a refractive index of the lithium niobate substrate;
a first electrode positioned adjacent to the waveguide; and
a second electrode positioned adjacent to the waveguide on an opposite side from the first electrode;
a light source optically coupled to the sensing device and positioned to deliver light into the waveguide;
an analytic module functionally coupled to the first and second electrode and operable to determine an electrical potential across the first and second electrodes;
a power source electrically coupled to the analytic module and to the first and second electrodes;
a first measurement electrode and a second measurement electrode electrically coupled respectively to the first and second electrodes, and operable to relay electric field measurements from an environment external to the system to the first and second electrodes; and
an indicator functionally coupled to the analytic module and operable to indicate the electric field measurements to a user.

17. The system of claim 16, wherein the analytic module is operable to quantify the electric field measurements.

18. The system of claim 16, wherein the indicator is a display screen.

19. The system of claim 16, wherein the first and second measurement electrodes are dry contact electrodes.

20. The system of claim 16, wherein the first and second measurement electrodes are contactless electrodes.

21. The system of claim 16, wherein the first and second measurement electrodes are operable to functionally couple to a biological entity.

22. The system of claim 21, wherein first and second measurement electrodes are operable to relay electric field measurements from an electric field generated by a biological system of the biological entity.
